(19) United States
(12) Patent Application Publication
(10) Pub. No.: US 2022/0288349 A1
(43) Pub. Date: Sep. 15, 2022

(54) NEONATAL CARE SYSTEM WITH SLING SLEEP DEVICE

(71) Applicant: GE Precision Healthcare LLC, Wauwatosa, WI (US)

(72) Inventors: Steven M. Falk, Baltimore, MD (US); Rajendra Naik, Bengaluru (IN); Helge B. Klockow, Wauwatosa, WI (US); Nagapriya Kavoori Sethumadhavan, Bengaluru (IN); Randeep S. Dang, Madison, WI (US); Ramune Auzelyte, Milwaukee, WI (US)

(73) Assignee: GE Precision Healthcare LLC, Wauwatosa, WI (US)

(21) Appl. No.: 17/831,171

(22) Filed: Jun. 2, 2022

Related U.S. Application Data
(63) Continuation-in-part of application No. 16/273,607, filed on Feb. 12, 2019, now Pat. No. 11,364,167.

(51) Int. Cl. A61M 21/02; A61G 11/00; A61F 7/00

(52) U.S. Cl. CPC A61M 21/02; A61G 11/00; A61F 7/007; A61M 2021/0066

(57) ABSTRACT

A bassinette for a neonatal care system includes a frame structure with at least one vertical a support member extending downward from a top support member. A sling is suspended from the top support member and configured to support a neonate. The sling comprises a pressure-diffusing netting material extending from a top end to a bottom end between opposing lateral sides. A heating coil may be configured to heat the neonate supported on the sling, and a sling controller may be secured to the frame and configured to selectively control the heating coil to heat the neonate. Additionally, a plurality of stretchable conductive electrodes may extend through the pressure-diffusing netting material and may be configured to acquire physiological signals from the neonate. The stretchable conductive electrodes may be configured to communicate the physiological signals from the plurality of conductive electrodes to a sling controller.

### CROSS REFERENCE TO RELATED APPLICATION

This application is a continuation-in-part of U.S. patent application Ser. No. 16/273,607, filed Feb. 12, 2019, the contents of which is hereby incorporated by reference in entirety.

### BACKGROUND

The present disclosure generally relates to neonatal care systems, and more specifically to neonatal care systems incorporating a sleep device having a sling and frame structure for supporting a neonate.

Some neonates and especially some significantly prematurely born infants are not physiologically well enough developed to be able to survive without special medical attention. A frequently used medical aid for such infants is the incubator. The primary objective of the incubator is to provide an environment which will maintain the neonate at a minimum metabolic state thereby permitting as rapid physiological development as possible. Neonatal incubators create a microenvironment that is thermally neutral where a neonate can develop. These incubators typically include a humidifier and a heater and associated control system that controls the humidity and temperature in the neonatal microenvironment. The humidifier comprises a device that evaporates an evaporant, such as distilled water, to increase relative humidity of air within the neonatal microenvironment. The humidifier is typically controllable such that the amount of water, or water vapor, added to the microenvironment is adjustable in order to control the humidity to a desired value. The heater may be, for example, an air heater controllable to maintain the microenvironment area to a certain temperature. Radiant warmers may be used instead of incubators for some neonates where less environmental control is required. In still other embodiments, hybrid incubator/radiant warming systems may be utilized, various embodiments of which are well known in the art.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one embodiment, a neonatal care system includes a base, a bassinet platform supported on the base, and a neonatal sleep device. The neonatal sleep device includes a frame structure connected to and extending upward from the bassinet platform and a sling suspended above the bassinet platform and configured to support a neonate. The neonatal care system further includes a heater configured to heat an environment surrounding the neonate supported in the sling.

One embodiment of a neonatal sleep device for a neonatal care system includes a frame structure configured to connect to a bassinet platform of a neonatal care system and a sling configured to support a neonate. The sling is removably attached to the frame structure such that it is suspended above the bassinet platform. The frame structure is collapsible so as to lower the sling and the neonate onto the bassinet to enable performance of medical care on the neonate.

An embodiment of a neonatal sleep device may include or be configured as a bassinette. A bassinette for a neonatal care system may include a frame structure with at least one vertical a support member extending downward from a top support member. A sling may be suspended from the top support member and configured to support a neonate. The sling may comprise a pressure-diffusing netting material extending from a top end to a bottom end between opposing lateral sides. A heating coil may be configured to heat a neonate supported on the sling, and a sling controller may be secured to the frame and configured to selectively control the heating coil to heat the neonate.

An embodiment of a sling may be configured to support a neonate in neonatal care system. The sling may include a pressure-diffusing netting material extending from a top end to a bottom end between opposing lateral sides. The top end, the bottom end, and the lateral sides may be configured to be removably attached to a bassinette frame of the neonatal care system. A plurality of stretchable conductive electrodes may extend through the pressure-diffusing netting material and may be configured to acquire physiological signals from the neonate. The stretchable conductive electrodes may be configured to communicate the physiological signals from the plurality of conductive electrodes to a sling controller.

Various other features, objects, and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a perspective view of an exemplary neonatal care system comprising a sleep device with a sling configured to support a neonate according to one embodiment of the present disclosure.

FIG. 2 is one embodiment of a neonatal sleep device for incorporation in a neonatal care system, such as an incubator or radiant warmer.

FIGS. 3A and 3B depict a neonatal sleep device according to another embodiment of the present disclosure.

FIGS. 4A-4C depict a neonatal sleep device according to another embodiment of the present disclosure.

FIGS. 5A-5C depict a neonatal sleep device according to still another embodiment of the present disclosure.

FIG. 6 depicts a neonatal sleep device according to another embodiment of the present disclosure that includes a tray for connecting to a neonatal care system.

FIGS. 7A-7D depict a neonatal sleep device according to another embodiment of the present disclosure.

FIG. 8 depicts one embodiment of a neonatal sleep device having a raisable portion of the bassinet platform according to one embodiment of the disclosure, and also including a phototherapy device on the bassinet platform and under the neonatal sleep device according to another embodiment of the present disclosure.

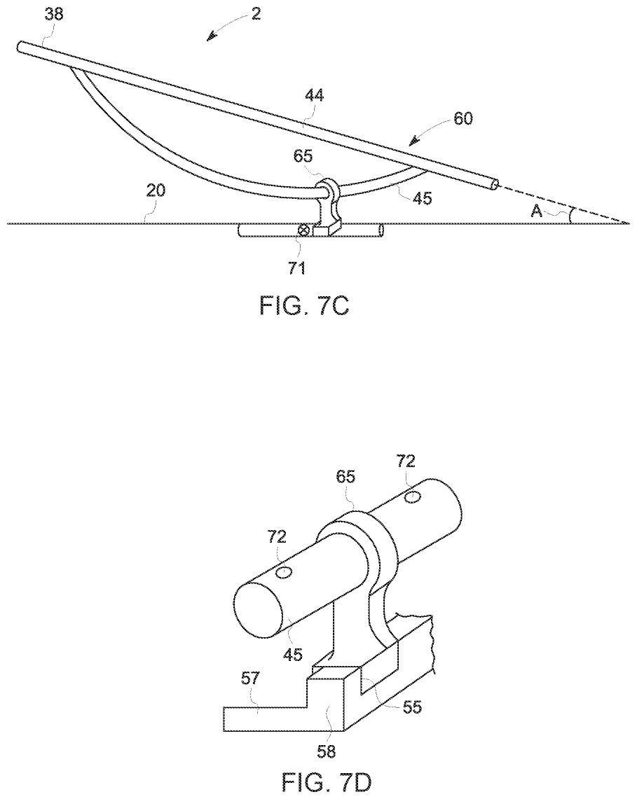

FIG. 9 depicts a cross-sectional view of a neonatal sleep device according to one embodiment of the disclosure.

FIG. 10 depicts an embodiment of a neonatal sleep device with a collapsible cover, handle, and a heating coil.

FIG. 11 depicts an embodiment of a neonatal sleep device with a collapsible cover and handles.

FIG. 12 depicts an embodiment of a neonatal sleep device with a plurality of heating coils.

FIG. 13 depicts an embodiment of a neonatal sleep device with an array of sensor electrodes.

FIG. 14 depicts a cross-section of the mesh layer of a sling including integrated electronics.

FIG. 15 depicts a block diagram of a neonatal sleep device including a plurality of sensor electrodes and a heating coil.

### DETAILED DESCRIPTION

The inventor has recognized that an improved neonatal sleep device is needed for neonatal care systems—such as incubators, radiant warmers, and other types of neonatal care stations and devices—including an improved pressure diffusing sleep surface that is breathable and allows administration of phototherapy to the neonate's entire body without touching the neonate, as well as integrated heating and patient monitoring of the neonate in such a way that is easily transportable and comfortable for the neonate. The inventors have recognized that existing bassinet mattress surfaces are often either too hard or do not provide sufficient breathability, such as existing foam mattress products and water mattress products available for neonatal care systems. Additionally, the inventors have recognized problems with existing bassinet platforms in neonatal care systems where misuse occurs too easily, such as placing babies on unclean or not adequately prepared mattress surfaces, and that systems should be developed to prevent such misuse cases. For example, a mattress may be installed incorrectly, or the incorrect mattress used in an incubator. Another misuse occurs where neonates are placed directly on a mattress without any sheet, or where an unclean or wrong sheet is placed on the mattress. Additionally, the inventors have recognized a need to an improved system that enables easy transport of the neonate between incubator or warmer systems, where patient monitoring and environmental temperature regulation of the neonate can continue during transport.

Through significant research and experimentation, the inventors developed the disclosed neonatal sleep device for a neonatal care system which provides improved pressure diffusion for supporting the neonate, while also providing a breathable sleep surface that permits air to circulate around the neonate. The disclosed neonatal sleep device includes a frame structure that connects to a bassinet of a neonatal care system, such as within a microenvironment of an incubator or under a radiant heater of a radiant warmer. A sling sleep surface and device comprising a pressure diffusing netting material attached to the frame structure and configured to support a neonate such that the neonate is suspended above the bassinet platform. The disclosed sling sleep device may be configured as a smart sling including pressure diffusing netting material with integrated features for acquiring physiological signals and/or measurements from the neonate and/or maintaining a desired environment for the neonate. The netting material of the sling may be formed of or include integrated conductive fibers that can be configured to perform environmental control and/or patient monitoring functions, such as conductive fibers configured as heating coils and controllable as part of the environmental control system of the neonatal care system and/or conductive fibers configured as electrodes and forming part of a physiological sensing system for the patient.

In certain embodiments, the sling sleep surface is formed of one or more materials that permit delivery of phototherapy. Namely, the sling is configured to permit blue light rays emitted by a phototherapy device, such as a blue LED phototherapy unit, to penetrate the sleep surface and reach the neonate's skin. The sling may be comprised of a netting material, for example, that is sufficiently stretchy to provide a comfortable and pressure diffusing sleeping surface, and also to absorb shock due to motion of the neonatal care system, thereby providing a smoother and more comfortable ride for the neonate during transport. The sling sleep surface provides the neonate a natural and relaxed sleep position, providing even pressure disbursement and minimizing the pressure points. The neonatal sleep device may also be adjustable so as to adjust the tilt angle of the sleep surface for the neonate.

In certain embodiments, the neonatal sleep device is collapsible so as to lower the sling and the neonate onto the bassinet platform to enable performance of medical care on the neonate. Namely, the pressure absorbing and/or bouncy sling may not be ideal for supporting a neonate during many medical procedures or examinations. And thus, the neonatal sleep device may be configured to allow the neonate to be lowered onto the firmer bassinette platform for performance of such medical care.

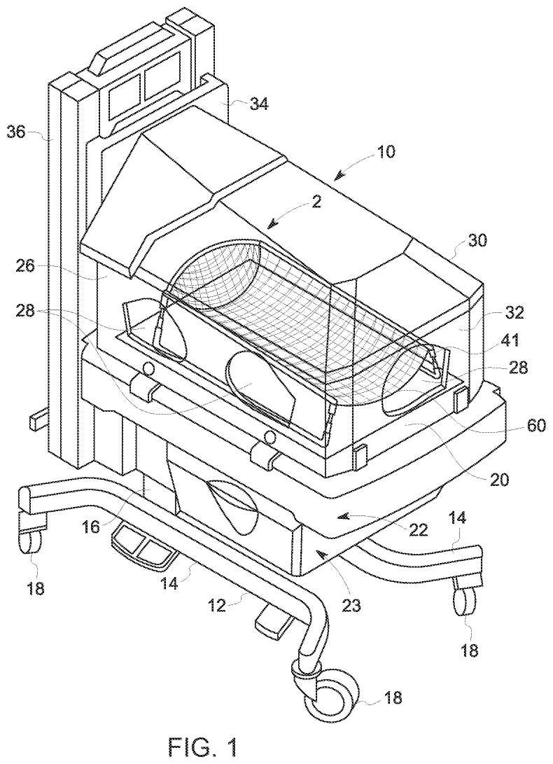

FIG. 1 depicts one embodiment of a neonatal care system 10 incorporating one embodiment of a neonatal sleep device 2. In the depicted embodiment, the neonatal care system 10 is an incubator; however, it should be appreciated that other types of neonatal care systems 10 may incorporate the neonatal sleep device 2, including radiant warmer systems, hybrid incubator/warmer systems, and standard bassinets for neonatal care environments without warming or environmental control systems. The depicted incubator neonatal care system 10 includes a base 12 comprising a pair of u-shaped horizontal members 14 joined together and providing support for a vertical base member 16. Wheels 18 may provide for ready movement of the neonatal care system 10. A bassinet platform 20 is supported on the vertical base member 16 of the base 12. In certain embodiments, the bassinet platform 20 may be a standard platform for supporting a neonate, such as configured to receive a removable mattress or other sleep surface in addition to the disclosed neonatal sleep device 2.

The neonatal sleep device 2 includes a frame structure 41 and a sling sleep surface 60 supported on the frame structure 41. The frame structure 41 connects to the bassinette platform 20 and extends upward therefrom. The frame structure 41 may be configured to removably connect to the bassinet platform 20. The sling 60 may be configured to removably attach and detach from the frame structure 41 such that, when attached, the sling 60 is suspended above the bassinette platform 20. The sling 60 may be a reusable element, such as comprised of washable material. In other embodiments, the sling 60 may be a single-use device that is disposable and configured for use with only a single neonate. In either embodiment, the sling 60 is configured to be removed from the frame structure 41 after each use with a neonate. Thus, the above-described misuse cases are prevented because the neonatal sleep device 2 requires placement of a clean and/or new sling sleep surface 60 prior to use. Moreover, the sling 60 may be shaped and configured such that it cannot be improperly attached to the frame 41, thereby preventing the misuse case of placing a neonate on an unclean or improperly configured sleep surface.

A hood 30 may enclose a chamber above the platform 20 to form a microenvironment 32 wherein temperature and humidity are controlled according to the needs of the neonate. The hood 30, when positioned and attached above the bassinet platform 20 includes a plurality of walls 26, normally of a transparent plastic material, which surround and enclose the neonate in the microenvironment 32. The walls 26 may have hand holes 28 to enable a caregiver to reach the neonate. Alternatively or additionally, at least one of the sidewalls 26 may be removable or hinged to open to provide better access to the neonate. Alternatively, a top portion of the hood 30 may be removable to provide complete access to the neonate. In still other embodiments, the hood 30 may be entirely removable from the platform 20. The hood 30 may be configured to abut the vertical frame members 36 such that the hood encloses three sides and the top of the microenvironment 32 and the vertical frame member 36 encloses the fourth side to maintain the microenvironment 32. In other embodiments, the hood 30 may define the entire enclosure above the bassinet platform 20.

The neonatal care system 10 may be configured such that the bassinet platform 20 can be raised and lowered, such as by having an extendible vertical base member 16 that is movable to adjust the height of the bassinet platform 20. The bassinet platform 20 may be mounted in a cantilevered manner such that various systems and devices can be mounted underneath. For example, a heater 22 may be mounted beneath or incorporated below the platform 20 and vents may be provided to circulate heated air into the microenvironment 32 defined by the hood 30. Similarly, a humidifier 23 may be incorporated below the platform 20 and controllable to evaporate water stored in a reservoir in order to control the humidity of the microenvironment 32. The heater 22 and humidifier 23 are controllable, such as by a control unit integrated into the neonatal care system 10, in order to control the humidity and temperature of the microenvironment 32.

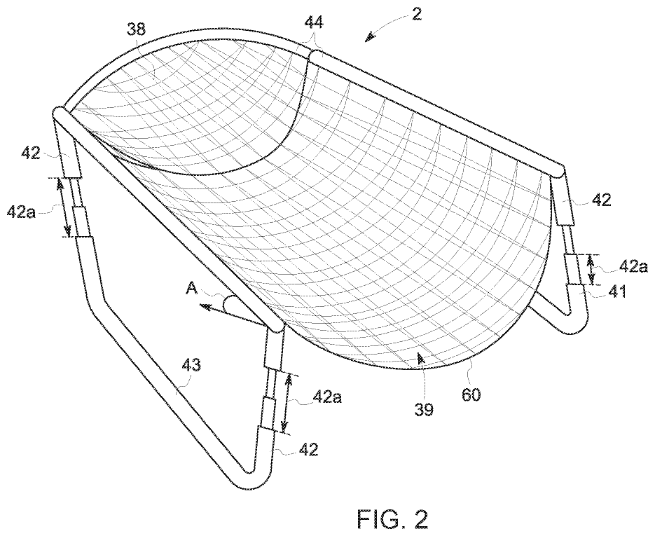

In the exemplary embodiment depicted in FIGS. 1 and 2, the neonatal sleep device 2 has a frame 41 comprised of vertical support members 42, horizontal support members 43, and at least one top support member 44. The horizontal support members 43 may connect to the bassinet platform 20, such as via any of the various connection means described herein. For example, the horizontal support members 43 may have clips, locking members, or other connectors that removably attach at connection points, or connection elements, on the bassinet platform 20. In other embodiments, the frame structure 41 may be permanently connected to the bassinet platform 20. In such an embodiment, the horizontal support member 43 may be permanently connected and/or integrated with the bassinet platform 20. In other embodiments, the vertical support members 42 may extend directly from the bassinet platform 20 and the horizontal support member 43 may be eliminated.

The top support member 44 is configured to attach to and detach from the sling 60, which is preferably removable from the frame structure 41. Various attachment means for attaching the frame and sling 60 are described herein. In one embodiment, the top support member 44 may be a straight bar on either side of the sling 60 connecting between the vertical support members 42 on the respective side. In other embodiments, the top support member 44 may be a continuous u-shaped rod, wherein the sling 60 is shaped to form over or otherwise connect to the top support member 44.

The vertical support members 42 may be extendible and retractable to raise and lower the height of the sling 60 above the platform 20. In certain embodiments, all four vertical support members 42 are vertically extendible, such as each having a telescoping portion 42a that can be extended or collapsed to raise and lower the sling. In other embodiments, only two out of the four vertical support members 42 may be vertically extendible so as to adjust the tilt angle A of the sleeping surface provided by the sling 60 with respect to horizontal. For example, the vertical support members 42 near the head-side of the neonate may be extendible upward so as to raise the head portion of the sling 60 to increase the angle A from horizontal. In other embodiments, the vertical support members 42 on the bottom side of the neonate may be collapsible in order to lower the foot portion 39 of the sling 60. In either embodiment, the angle A of the sleep surface provided by the sling 60 may be adjustable to positive or negative angles (e.g., a Trendelenburg position).

In certain embodiments, frame structure 41 may be collapsible to allow the sling 60 supporting the neonate to lower all the way to the bassinet platform 20. This may be desirable to enable performance of medical care on the neonate where the stretchable sling 60 may not provide an ideal surface for supporting the neonate. For example, the vertical support members 42 may be configured be shortened sufficiently to lower the sling 60 such that the entire weight of the neonate is supported on the platform 20. In such an embodiment, the telescoping portions 42a of the vertical support members may be configured to allow the vertical support members 42 to become small enough such that the sling 60 contacts the bassinet platform 20 and the bassinet platform 20 supports the weight of the neonate. In other embodiments, the bassinet platform 20, or a portion thereof, may be configured to be raisable so as to support the neonate to enable performance of certain medical care for which the sling sleep surface 60 is not ideal.

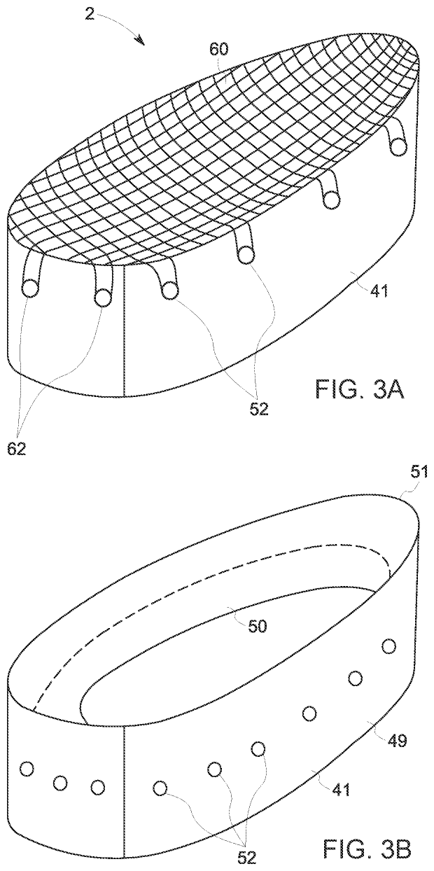

In other embodiments, the frame 41 may be fixed such that the height of the frame is not adjustable. FIGS. 3A and 3B depict one such embodiment, where the frame 41 is a continuous support structure surrounding the sling 60. In the example, the frame 41 includes a vertical exterior side 49 and a ramped interior side 50, which join at a top lip 51. The sling 60 stretches over the top lip 51 and attaches to points on the exterior side 49 of the frame 41. In other embodiments, the exterior side 49 may be ramped and the interior side 50 may be vertical. In still other embodiments, both the interior and exterior sides may be vertical, or both may be ramped. The frame 41 may be sized and shaped to pair with the bassinet platform 20, such as to fit snugly within a groove or tray of the platform. To provide one example, the groove or tray may be alternatively used for containing a mattress when not in use with the neonatal sleep device 2.

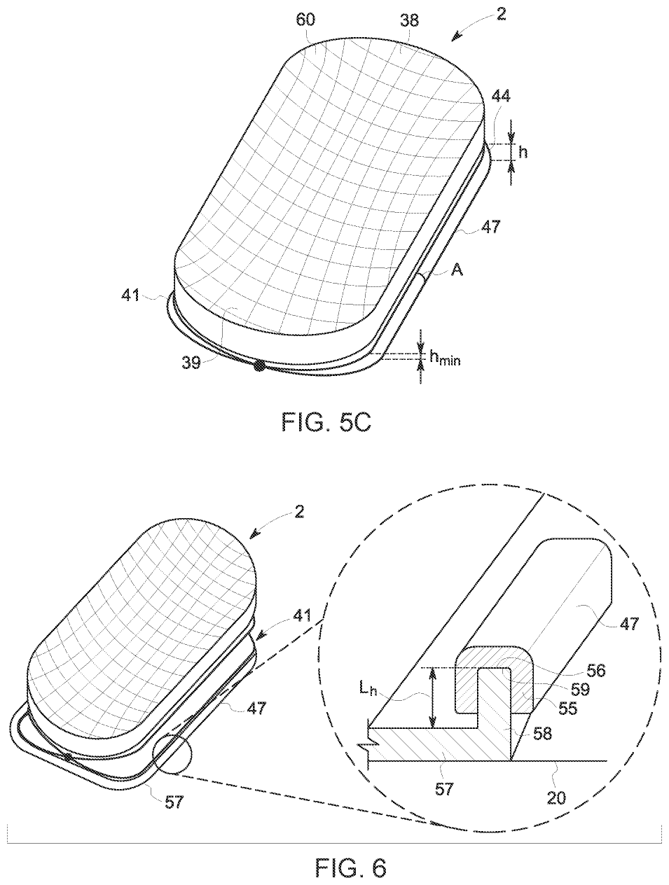

Various attachment means may fixedly secure the frame structure 41 to the platform 20. In certain embodiments, the frame 41 and/or the bassinet platform 20 may comprise corresponding elements configured to mateably connect, such as clips, pins, and holes, hooks, locks, or other fasteners that secure the frame 41 to the bassinet platform 20. Still other friction-fit attachment means may connect the frame structure 41 and the bassinette platform 20 (see FIG. 6 and description below). In other embodiments where the neonatal sleep device 2 is not removable and is permanently fixed to the bassinet platform 20. For example, the frame 41 may comprise a continuous piece with a portion of the bassinet platform 20.

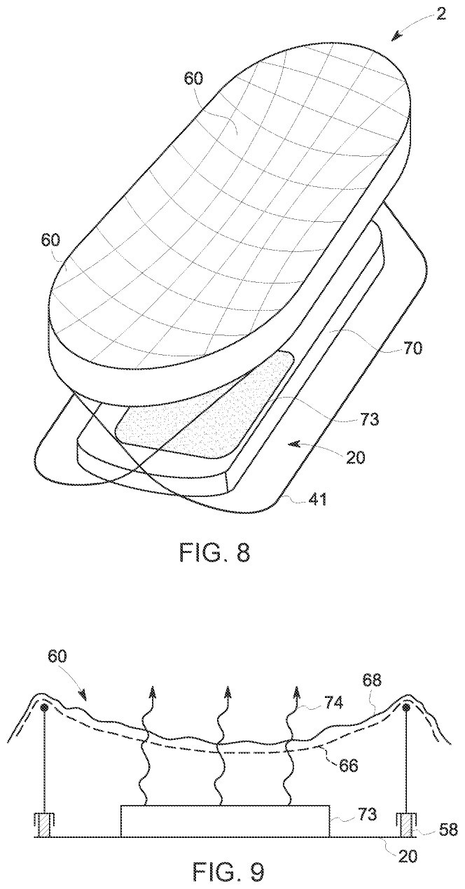

The frame structure 41 and sling 60 may attach together by various means. In the example, the sling 60 attaches at various attachment points around the frame 41. The frame 41 comprises sling attachment means 52 for attaching to the sling 60. The sling comprises corresponding frame attachment means 62 for attaching to the frame 41. For example, the sling attachment means 52 may be hooks or buttons, and the frame attachment means 62 may be loops or strips with eyelets configured to attach to the sling attachment means 52. To provide just one example, the sling 60 may comprise a mesh layer comprised of a netting material 66 (see FIG. 9), and the mesh layer may be configured to loop over the sling attachment means 52, thereby providing the frame attachment means 62. The mesh layer of the sling 60 connected to the sling attachment means 52 is thereby configured to support the weight of the neonate. For example, the frame attachment means 62 on the sling 60 may be snaps, clips, pins, hooks, holes, or other elements that mate with corresponding sling attachment means 52 so as to fasten the sling 60 on the frame 41. Still other friction-fit frame attachment means 62 and sling attachment means 52 may be provided, such as where the top support member 44 or the top lip 51 of the frame structure 41 comprises a clamp that clamps over the sling 60 so as to provide connection between the frame structure 41 and the sling 60.

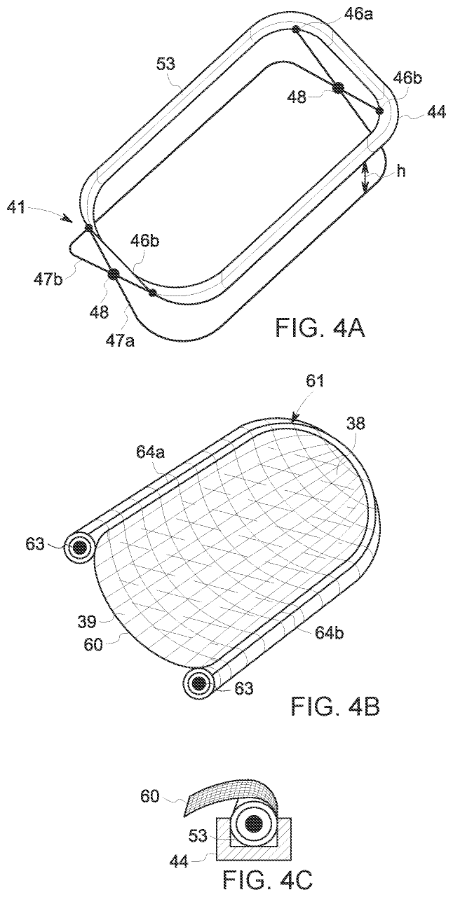

FIGS. 4A through 4C depict another embodiment of the neonatal sleep device 2 where the frame 41 has a connection means provided on the top support member 44. Referring to FIGS. 4A and 4C, the top support member 44 includes a groove 53 configured to receive a sling frame member 63. In the example, the sling frame member 63 is a rod sized to fit within the groove 53 on the top support member 44 of the frame structure 41. FIG. 4C is a cross-sectional view of the connection. The sling frame member 63 connects to the material of the sling 60. As illustrated in FIG. 4B, the material of the sling 60 may wrap around the sling frame member 63, which in this example is a rod, and the wrapped sling frame member 63 fits within the groove 53 of the top support member 44. Thereby, the sling 60 is maintained in place by the tight connection between the sling frame member 63 and the groove 53. In one embodiment, the sling 60 is a separate material piece from the sling frame member 63, where the sling frame member slides in and out of a channel sewn or otherwise formed along the outer edge 61 of three sides of the sling 60. Thereby, the sling is configured to mate with the top support member 44 along three sides of the sling 60. In other embodiments, only two lateral sides of the sling 60 may only attach to the top support member 44 along the two lateral sides 64a, 64b. In such an embodiment, the sling frame member 63 may comprise two lateral side portions, such as two rods, that connect in corresponding grooves on the lateral sides of the top support member 44.

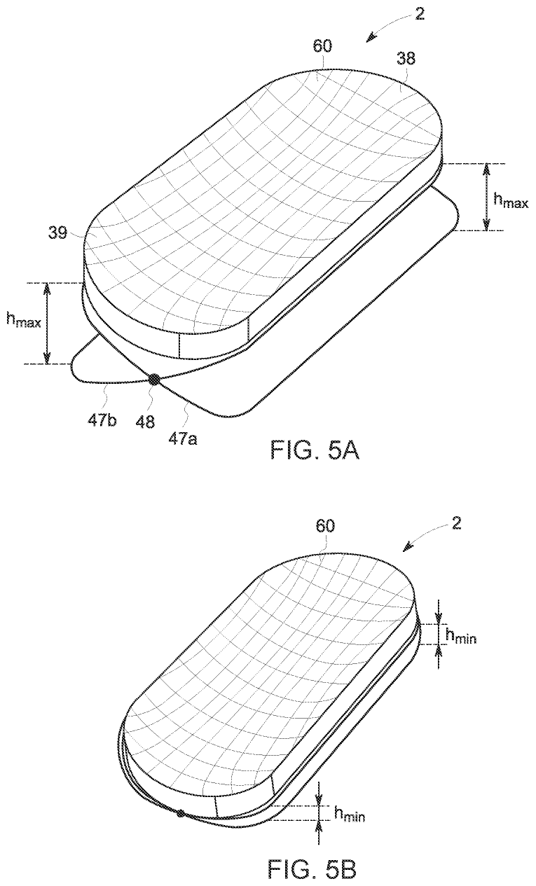

FIGS. 4A and 5A-5C depict an embodiment of a collapsible frame structure 41 comprising a top support member 44 supported on two diagonal-crossing support members 47a and 47b that are connected at a movable joint 48 such that the frame 41 can be raised and lowered. The support members 47a and 47b connect to the top support member 44 at a respective top joint 46a, 46b as shown in FIG. 4A. In one embodiment, the movable joint 48 may be configured to maintain and control the relative position of the support members 47a and 47b so as to control the height h of one or both end portions 38, 39 of the sling 60. FIGS. 5A-5C exemplify various heights and tilt angles that may be achieved with the collapsible frame structure 41. In FIG. 5A, the supports 47a and 47b are positioned at a maximum height hₘₐₓ. Thereby, the sling 60 is raised to a maximum height above the bassinet platform 20. FIG. 5B depicts the frame 41 fully collapsed, and thus at a minimum height hₘᵢₙ. Thus, the support members 47 move with respect to one another via the movable joint 48 to raise and collapse so as to raise and lower the sling 60 with respect to the bassinet platform 20. In certain embodiments, the movable joint 48 and the support members 47 may be configured to be raised to achieve multiple heights between the minimum hₘᵢₙ and the maximum hₘₐₓ.

In certain embodiments, the frame 41 may be configured such that the ends may be positioned at different heights so as to control a tilt angle of the sleeping surface for the neonate. FIG. 5C provides one example where the foot portion 39, or the end of the sling 60 adjacent to the feet of the neonate, is at a lower height than the head portion 38 of the sling 60. In the example at FIG. 5C, the foot portion 39 is at a minimum height hₘᵢₙ, such that the movable joint 48 and support members 47 are fully collapsed on the foot side of the frame 41. The head portion 38 is raised to height h so as to provide tilt angle A for supporting the sling 60 and a tilted sleeping surface for the neonate. The head portion 38 may be raised and lowered to increase and decrease the tilt angle A, such as by adjusting the movable joint 48 and support members 47a and 47b on the side of the head portion 38, to provide an adjustable sleep angle for the neonate. Likewise, the tilt angle A can be adjusted by adjusting the height at the foot portion 39.

FIG. 6 depicts one way that the neonatal sleep device 2 may be configured to removably connect to the bassinet platform 20. In one embodiment, a bottom portion of the frame structure 41 may comprise clips configured to clip to a corresponding portion of the platform 20 or some element provided on the platform 20. In the example, the bottom portion of the support members 47 form a clip 55 having a groove configured to provide a pressure fit over a lip 58 extending above the bassinet platform 20. For example, the clip 55 may include a deformable material inside the groove, such as rubber or softer plastic, so as to provide a friction fit over the lip 58. Thereby, the neonatal sleep device 2 can be securely connected to the bassinet platform 20. For example, the lip may extend from a tray 57, which may comprise part of the bassinet platform. The tray 57 may be formed to also contain a bassinet mattress, which may be an alternative to or used in conjunction with the neonatal sleep device 2. Alternatively, the tray 57 may be separable from the remainder of the bassinet platform 20, such as when the neonatal care system 10 is in use without the neonatal sleep device 2. Accordingly, the tray 57 may provide part of the connection means for removably connecting the frame structure 41 to the bassinet platform 20.

In other embodiments, the lip 58 may extend and be integrated into the bassinet platform 20, rather than have a tray 57. FIG. 9 exemplifies such an embodiment, where the lip 58′ extends from an integral part of the bassinet platform 20. Other fastening mechanisms, or clips, may be provided for connecting the frame 41 to the bassinet platform, such as locking mechanism, hooking devices, etc. The lip 58 has a height Lₕ, which may extend above the inside of the tray 57 above the bassinet platform 20. The clip 55 is configured such that an interior top side 56 of the clip 55 meets the top 59 of the lip 58. Thereby, the frame structure 41 can be steadily and securely supported on the bassinet platform 20. FIG. 6 depicts an embodiment where the bassinet platform 20 further comprises a raised platform portion 70. In certain embodiments, the raised platform portion 70 may be a fixed and permanent part of the bassinet platform 20 or may be removably attachable. The raised platform portion 70 may be configured to receive and support the neonate when the frame structure 41 is fully collapsed, so as to enable performance of medical care on the neonate by providing a steady support surface. In other embodiments, the raised platform portion 70 may be configured to be raised and lowered, so as to enable bringing the platform surface upward to contact the sling 60. Thus, the bassinet platform 20 (or a portion or section thereof) may be raised, rather than the frame structure 41 collapsing, in order to support the neonate on a flat and sufficiently stable surface to enable performance of medical care.

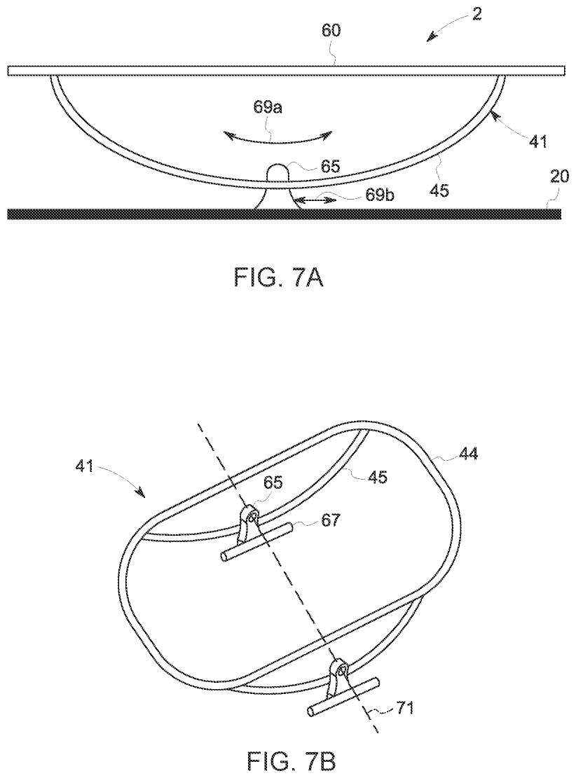

FIGS. 7A-7D depict another exemplary embodiment of the neonatal sleep device 2 where the frame 41 is comprised of a curved support member 45 moveable with respect to a connector 65 that provides connection to the bassinet platform 20. In the depicted embodiment, the frame 41 has a curved support member 45 connecting to and supporting a top support member 44 that, in turn, connects to and supports the sling 60 (such as via the grooves on the lateral side as shown above). The curved support member 45 is arched to support the sling 60 at a vertical height above the platform 20, and the curved support member 45 may be configured of a material that permits flex so as to provide additional shock absorption.

The curved support member 45 may be reciprocally designed with the bassinet connector 65 such that the curved support member 45 is moveable with respect to the bassinet connector 65 in order to adjust the tilt angle A for supporting the sling 60 and providing an angled sleeping surface for the neonate. In the depicted embodiment, the curved support member 45 is moveable in the fore and aft directions indicated by arrow 69a. The connector 65 may also be moveable with respect to the bassinet platform 20, as exemplified by arrow 69b, so as to compensate for at least a portion of the directional movement of the curved support member 45. Namely, the connector 65 may move in an opposite direction from the curved support member 45, thus a change in the relative position of the sling 60 with respect to the head-side or foot-side of the microenvironment can be minimized.

FIGS. 7A and 7B depict the frame 41 in a centered position, where the sleep surface is horizontal and parallel with the bassinet platform 20. The connector 65 is in a centered position along a centered horizontal line 71. FIG. 7C depicts a tilted position, where the head portion 38 of the sling 60 is raised and a foot portion 39 is lowered so as to position the frame 41 supporting the sling 60 at angle A. The connector 65 is moved away from the center line 71 toward the foot-side, which compensates for at least some of the movement of the sleep surface toward the head-side of the neonatal care system 10. In certain embodiments, the connector 65 may connect to a slideable track 67 or other adjustable device integrated into or otherwise securely connected to the bassinet platform 20. In other embodiments, the neonatal sleep system 2 may comprise a tray 57 as shown in FIG. 7D and described above with respect to FIG. 6. For example, the connector 65 may comprise a clip 55 that connects to a lip 58 of the tray 57. In such an embodiment, the connector 65 may be moved lengthwise along the lip 58 in order to position the neonatal sleep device 2 appropriately within the neonatal care system 10.

In the depicted embodiment, the curved support member 45 is moveable with respect to the connector 65 and is lockable at certain predefined positions so as to change the tilt angle A of the neonatal sleep system 2. In the depicted embodiment at FIG. 7D, the curved support member 45 has holes 72 that receive a spring-loaded locking pin or other portion in the connector 65 that inserts into the holes 72 in order to lock the curved support member 45 in place. In other embodiments, the connector 65 and curved support member 45 may have other connecting means, which may provide continuous connection along any part of the curved support member 45 or connection at particular locations (such as at the locations of the holes 72 in the depicted example).

In certain embodiments, a phototherapy device 73 may be placed underneath the sling 60 so as to provide phototherapy to the neonate. In such an embodiment, the sling 60 is comprised of a material that permits delivery of phototherapy therethrough—i.e., that is penetrable by blue wavelength light emitted by the phototherapy device 73. FIG. 9 depicts an example where the phototherapy device 73 is positioned beneath the sling 60, and wherein the blue light 74 emitted by the phototherapy device 73 penetrate through the sling 60 so as to reach the neonate. Thereby, phototherapy can be provided to all (above and below the patient) sides of the neonate without touching the neonate or otherwise changing the neonate's position. Thus, as will be understood by a person having ordinary skill in the art in view of this disclosure, two or more light-emitting phototherapy devices may be positioned in the neonatal care environment so as to direct light toward the neonate from multiple directions, such as opposing directions. The sling 60 may be any material appropriate for supporting a neonate. In one embodiment, the sling 60 may be comprised of two layers, including a netting material 66 and a sheet top layer 68. The netting material may be a pressure-diffusing material. Netting material may be comprised of various non-latex materials having stretching and contracting properties, such as polypropylene, polyethylene, nylon, PVC or PTFE, or a similar synthetic, man-made polymer such as polyethylene terephthalate. It could be elastomeric fabric, such as a synthetic fabric providing either 2-way stretch or 4-way stretch, crosswise and/or lengthwise. The net pattern may vary, and may be, for example, from 50% to 90% open.

The netting material 66 may be covered by a sheet top layer 68, which is any material appropriate for contacting the skin of the neonate. The sheet 68 material may be constructed from materials that are partially translucent for light wavelengths of peak phototherapy device operation, such as for wavelengths in the range of 445 nm-470 nm. The assembled sleep surface comprised of netting material 66 and sheet 68 preferably provides low frequency vibration damping. In one exemplary embodiment, the maximum peak pressure measured by placing 680 g (1.5 lbs.) on the sling sleep surface does not exceed 28 mmHG and is capable of supporting up to 5 kg (11 lbs.) distributed weight on the sleep surface netting area while keeping it elevated at least 5 mm (0.2 inches) above the microenvironment platform 20. The sling 60, including the sheet and netting assembly, preferably meets X-ray image clarity requirements and will produce no artifacts in the X-ray image that make the X-ray difficult to read as evaluated by a radiologist. The netting material 66 and sheet 68 are preferably constructed from non-toxic, DEHP and BPA free, materials and not from materials known as potentially causing allergic reactions, such as materials having any animal tissues. The netting material 66 and sheet 68 preferably have neutral or low odor. The sheet 68 is preferably impermeable and not absorbent of any liquids. Preferably, any seams or other material-connecting areas of the netting material 66 and/or sheet 68 shall not come in contact with a neonate when the sling 60 is in use. As exemplified in FIG. 9, the sheet material 68 may be oversized, and configured with sufficient slack such that the weight of the neonate is supported by the pressure-diffusing netting material 66, rather than the sheet top layer 68.

Some embodiments of a neonatal care system may include a sleep device with a smart sling including integrated features for acquiring signals and measurements from the neonate and/or maintaining a desired environment for the neonate. The netting material 168 of the sling may be formed of or include integrated conductive fibers that can be configured to perform environmental control and/or patient monitoring functions. For example, the netting material 168 may include conductive fibers configured as heating coils and controllable as part of the environmental control system of the neonatal care system.

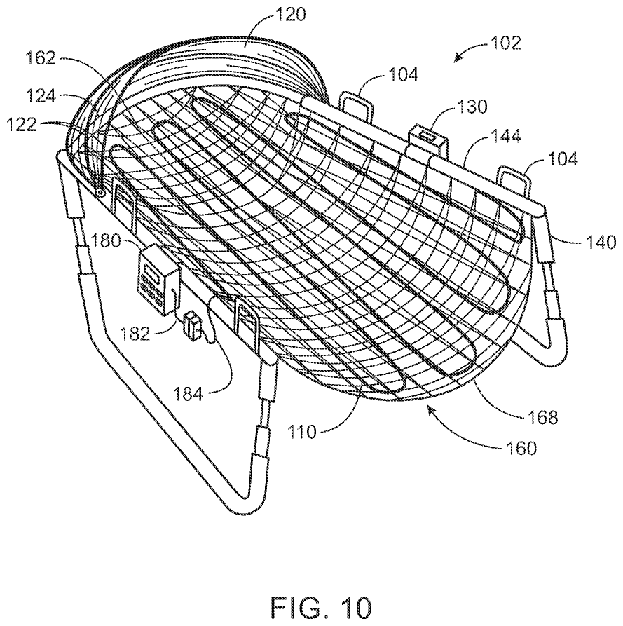

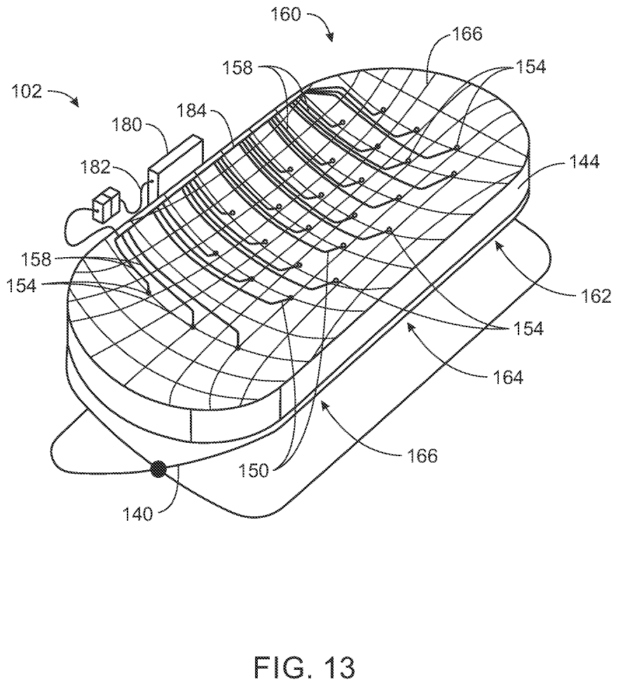

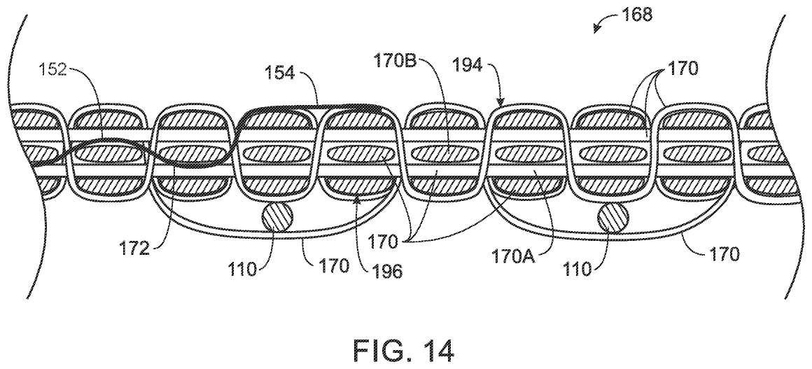

Alternatively or additionally, the netting material 168 may include conductive fibers configured as electrodes and forming part of a physiological sensing system for the patient. The electrodes may be configured to acquire various different physiological signals and measurements from the neonate, such as electrocardiogram (ECG) signals, respiration signals, electroencephalogram (EEG) signals, temperature measurements, and/or any other type of physiological signal or measurements from the neonate. Signals and measurements obtained by the electrodes may be communicated to a sling controller 180 configured to operate at least one of the environmental control system and the physiological sensing system. Amongst other functions, the sling controller 180 may be configured to receive signals and measurements from electrodes or other sensors of the sleep device; process the acquired signals or perform calculations using the acquired signals; communicate acquired signals and measurements and/or any processed data to other devices or systems; control the electrodes, heating coil, and/or any other system or device; and perform any other functions of the sleep device. FIGS. 10, 12 and 14 illustrate embodiments of a neonatal sleep device 102 including a frame 140 configured to support a sling 160 with an integrated heating coil(s) 110. FIGS. 13 and 14 illustrate embodiments of a neonatal sleep device 102 including a sling 160 with integrated patient electrodes fibers 158. These exemplary embodiments are discussed in further detail below.

As previously discussed, the sling 160 may comprise a pressure-diffusing netting material 168 formed with stretchable fibers to provide the neonate with a sleeping surface that evenly distributes pressure to minimize pressure points and absorbs shock due to motion of the sleep device 102 during transport. The sling 160 may be removably attachable to at least one top support member 144 of the bassinette frame 140 by attachment means that couple the sling 160 to the top support member 144 along at least one of a top end of the sling 160, a bottom end of the sling 160, and the lateral sides of the sling 160.

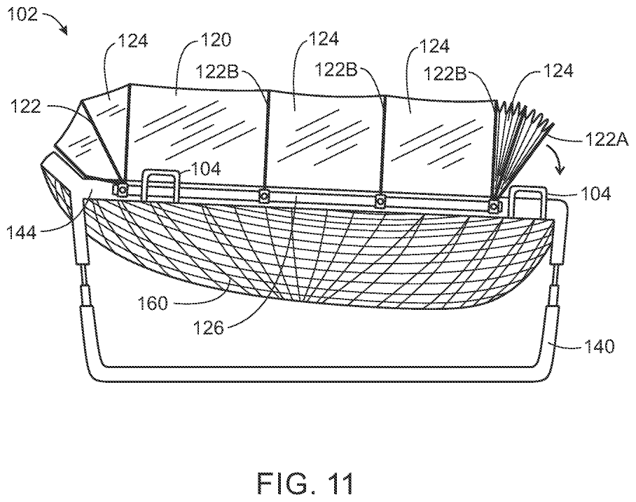

In some embodiments, the frame 140 may include features configured to assist in transporting the sleep device 102. As illustrated in FIGS. 10-11, a plurality of handles 104 may be coupled to the top support member 144. The handles 104 can serve as lifting points that may be used to carry the sleep device 102 and/or to hang or support the sleep device 102 in a neonatal care system. In the illustrated embodiments, the sleep device 102 includes two handles 104 on each lateral side of the top support member 144, and at least one of the handles 104 may be removably attached to the top support member 144.

Some embodiments of a sleep device 102 may include a cover or hood configured to enclose a neonate partially or completely on the sleep device 102 to maintain a microenvironment with desirable environmental conditions for the neonate. In the embodiment of FIG. 10, the sleep device 102 includes a collapsible cover 120 configured to enclose a head portion 162 of the sling 160 at the top end thereof. The collapsible cover 120 may be pivotably mounted on the top support member 144 and can include a plurality of folding rigid members 122 with a flexible cover material 124 extending between the rigid members 122. The cover 120 may be moved between a collapsed position in which the rigid members 122 are folded down onto the top support member 144 and an expanded position in which rigid members 122 are unfolded to pull the flexible cover material 124 over the head portion 162 of the sling 160. The remainder of the sling 160 may be enclosed by a removable cover (not shown) that may be attached to the top support member 144 and/or the sling 160 to fully enclose the sleeping area.

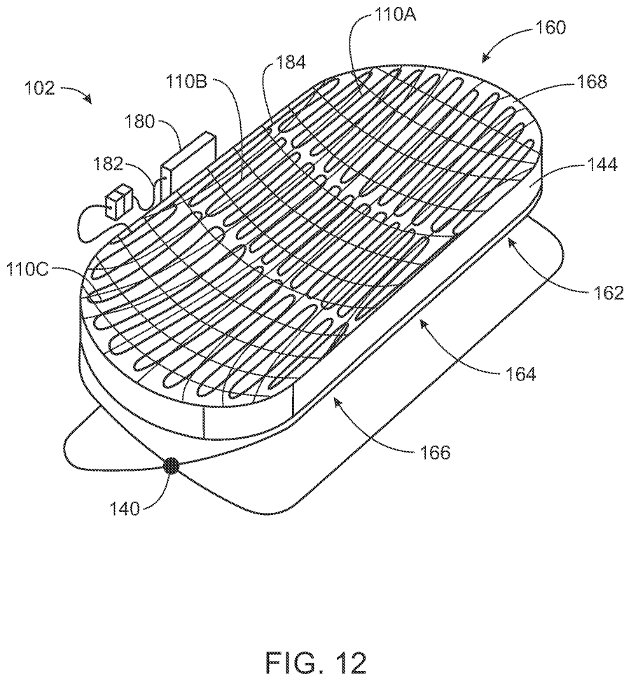

Some embodiments, however, may include an integrated cover attached to the frame 140 and configured to enclose the entire sling 160. As illustrated in FIG. 12, the sleep device 102 may include a collapsible cover 120 movable from a collapsed position to an expanded position in which the cover 120 extends from the top end of the sling 160 to the bottom end of the sling 160. The collapsible cover 120 may include a plurality of folding rigid members 122, and at least one of the rigid members 122 is configured slide along the lateral sides of the top support member 144 to pull the flexible material 124 over the entire sling 160. In the illustrated embodiments, a lead rigid member 122A is pivotably and rotatably mounted on the lateral sides of the top support member 144. As the lead rigid member 122A slides along a rail 126 towards the bottom end of the sleep device 102, the flexible cover material 124 is pulled over the sling 160. At least one intermediate member 122B may be pulled towards the bottom end by the flexible cover material 124 so that the intermediate member 122B provides support for the flexible cover material 124 between the top end and the bottom end of the sleep device 102. Once the lead rigid member 122A is positioned proximate the bottom end of the sleep device 102, it may be pivoted down such that it abuts the bottom end of the sling 160, thereby enclosing the sleeping surface provided by the netting material 168.

Some embodiments of a sleep device may include a differently configured cover. For example, a collapsible cover may be integrated into the frame of the sleep device, or it may be removably attached to the frame. Additionally or alternatively, while the illustrated covers are configured to fold an unfolded by moving longitudinally from the top end of the sleep device to the bottom end, some embodiments may fold in a different direction, for example laterally across the sling from one lateral side of the top support member to the opposite lateral side.

Some embodiments of a neonatal sleep device 102 may include mounting features configured to support various different medical devices and accessories on the frame 140. For example, referring to FIG. 10, at least one mounting support 130 may be configured to receive and support an accessory device (e.g., a power source, sensor(s), a support arm, etc.) on or near the top support member 144. The mounting supports 130 may be integrally formed in the frame 140, permanently attached to the frame 140, and/or removably attached to the frame 140. Other embodiments may omit a mounting support.

Embodiments of the sling 160 may include an integrated heating coil 110 configured to warm a neonate supported on the netting material 168. In the embodiment of FIG. 10, the heating coil 110 is arranged in a serpentine pattern that extends between lateral sides of the sling 160 from a first end to a second end. This may be useful, for example, so that the position of the heating coil 110 can be maintained as the netting material 168 stretches and contracts. Power may be supplied to the heating coil 110 by a power source mounted on at least one of the frame 140 of the sleep device 102 and the netting material 66 of the sling 160. In the illustrated embodiments, the heating coil is powered by a sling controller 180 that is attached to a lateral side of the top support member 144. A connector cable 182 may extend from the sling controller 180 and connects to a sling plug that may be positioned at the netting material 168, or that is at the end of a sling cable 184 extending from the sling 160.

In some embodiments, the sling cable 184 may extend along the lateral side of the sling 160 and can link a plurality of heating coils to the sling controller 180. For example, as illustrated in FIG. 12, the sling cable 184 extends along the side of the sling 160 proximate the top support member 144 and connects three separate heating coils 110A-C to the sling controller 180. Each of the heating coils 110A-C may be independently controllable to heat different sections of the sling 160. In the illustrated embodiment, a first heating coil 110A is configured to heat the head portion 162 of the sling, a second heating coil 110B is configured to heat a center portion 164 of the sling 160, and a third heating coil 110C is configured to heat the foot portion 166 of the sling. This may be useful, for example, in order to provide different amounts of heat to different parts of the neonate's body.

Embodiments of the heating coil(s) 110 may comprise a conductive wire, cable, and/or fiber that is supported by and/or woven into the netting material 168. As discussed in greater detail with respect to FIG. 14, the heating coil 110 may be a stretchable conductive fiber or wire that is woven into the non-conductive stretchable fibers of the netting material 168. Embodiments of a sleep device may use a wide variety of commercially available stretchable conductive fibers, such as Amotape textile electrodes from Amohr, Berkaet conductive textiles, Embro smart textiles, Holland Shielding Systems BV stretch conductive fibers, stretchable conductive inks, and/or and other suitable stretchable conductive material. Additionally or alternatively, the heating coil 110 may be a stretchable or non-stretchable conductive fiber or wire that is supported by the netting material 168. At least some of the non-conductive fibers of the netting material 168 may space the heating coil apart from the sleeping surface of the sling 160 so that the heating coil(s) 110 do not come into direct contact with the neonate. Further still, some embodiments of a heating coil may comprise a stretchable, conductive printed ink that is deposited on a fiber or fibers.

Some embodiments of a sleep device may include a differently configured heating arrangement. For example, a sling for a sleep device may include a different number of heating coils than in the illustrated embodiments, such as more than three heating coils. While the illustrated heating coils generally extend across the sling in a lateral direction, some embodiments may include at least one heating coil in a different orientation, and/or at least one heating coil that is not arranged in a serpentine pattern. Further still some embodiments may be configured without any heating coils.

In addition or as an alternative to a heating coil, some embodiments of a sling for a neonatal sleep device may include an array of sensors and/or electrodes configured to acquire physiological signals from a neonate resting on the sling 160. Referring to FIG. 13, the neonatal sleep device 102 may include a plurality of electrodes 150, 152 configured to transmit electrical potential and/or physiological signals that are acquired from the neonate to the sling controller 180. Each of the electrodes 150, 152 may include an electrode contact 154 (see, e.g., FIG. 14) configured to make contact with the neonate and an electrode wire or fiber 158 that extends through or is supported by the netting material 168 of the sling 160. As discussed in greater detail with respect to FIG. 14, at least one of the electrode wires 158 may be a stretchable conductive fiber or wire that is woven into the non-conductive stretchable fibers of the netting material 168. Additionally or alternatively, at least one of the electrode wires 158 may be a non-stretchable conductive fiber or wire that is supported by the netting material 168. Each electrode wire 158 extends from a corresponding electrode contact 154 to the sling cable 184 extending along the lateral side of the sling 160. Signals or electrical potential from each of the electrodes 150, 152 are transmitted to the sling controller 180 via the sling cable 184 and the connector cable 182. Additionally or alternatively, at least one of the electrodes 150, 152 may be connected directly to the sling controller 180 or the connector cable 182 by the electrode wire 158 of said electrode 150, 152.

In the illustrated embodiment, the sling 160 includes electrodes configured as temperature sensing electrodes 150 and ECG electrodes 152. The temperature electrodes 150 may be arranged in an array that extends across the head portion 162, center portion 164, and foot portion 166 of the sling 160 so that temperature measurements may be acquired from various different locations across the body of the neonate. Temperature information acquired by the temperature electrodes 150 is transmitted to the sling controller, which may be configured to process and/or utilize the collected measurements. For example, temperature measurements collected from the neonate may be used to create a temperature map of the neonate. This may be useful, for example, to control a heating system to maintain a desired body temperature of the neonate. In embodiments of the sling 160 that include temperature sensors 150 and at least one heating coil 110, the sling controller may be configured to control the heating coil 110 based on the collected temperature measurements. Furthermore, in embodiments that include a plurality of heating coils 110A-C, the processor may use the temperature measurements to individually control each of the heating coils 110A-C.

With continued reference to FIG. 13, the electrodes 152 may be arranged in array the extends across the head portion 162 and the center portion 164 of the sling 160. The sling controller 180 may be configured to dynamically utilize the ECG electrodes 152 to acquire ECG signals, identify a heartbeat, and determine a heart rate of the neonate. For example, the sling controller 180 may be configured to dynamically select two of the ECG electrodes 152 to use to acquire ECG signals from the neonate. This may be useful, for example, in order to acquire ECG signals with high signal strength. The sling controller 180 may select the two ECG electrodes 152 based on at least one of a signal-to-noise ratio, a signal stability, and any other signal characteristic or criteria. The sling controller 180 may be configured to continuously measure signal characteristics and select a pair of ECG electrodes 152 at regular intervals. In one example, a first one of the ECG electrodes 152 may be selected on one side of the neonate's heart while a second ECG electrode 152 is selected on the opposite side of the neonate. The sling controller 180 may be configured to use acquired signals from the non-selected ECG electrodes in order to filter out signal noise caused by movement of the neonate on the sling 160. Additionally or alternatively, the sling controller 180 may be configured to selectively power one of the ECG electrodes 152 for use as a drive electrode. This may be useful, for example, in order to reduce signal noise in the ECG signals. The sling controller 180 may dynamically select one of the ECG electrodes 152 for use as the drive electrode.

Some embodiments of a sleep device may include a sling with a different electrode configuration. For example, a sling may include only temperature electrodes or ECG electrodes. The illustrated sling 160 includes fifteen temperature electrodes 150 and twelve ECG electrodes. Some embodiments, however, may include a different number of temperature electrodes and/or ECG electrodes. Additionally or alternatively each electrode type may be arranged in an array having a different shape or size than in the illustrated embodiments. For example, a sling may include more than fifteen temperature electrodes 150 and twelve ECG electrodes arranged in tightly packed arrays for acquiring physiological signals or electrical potential from the neonate. While the illustrated electrodes each have an electrode wire that extends laterally across the sling to connect to the sling cable, some embodiments may include at least one electrode with a longitudinally extending electrode wire.

As previously mentioned, embodiments of a sleep device may include heating coils and/or electrode wires that are integrated into the material of the sling as stretchable or non-stretchable fibers or wires. Referring to FIG. 14, a detailed cross-sectional view of the netting material 168 of the sling 160 that may include stretchable and/or non-stretchable fibers 172, 174 is illustrated. The netting material 168 of the sling includes a plurality of non-conductive fibers 170 that are woven together to create a sleeping surface for supporting the neonate thereon. The non-conductive fibers 170 are arranged in layers that alternate between laterally and longitudinally extending fibers 170, and that provide a top surface 194 and a bottom surface 196 of the sling 160. While the nonconductive fibers 170 of the illustrated embodiment are woven in an angled interlock pattern, it should be appreciated that some embodiments may include non-conductive fibers a different weave pattern with at least one of a different number of layers, a different arrangement of fibers, different spacing between fibers, and differently sized fibers. In the illustrated embodiment, the sling 160 includes an electrode—exemplarily configured as an ECG electrode 152 with a contact surface 154 and a stretchable conductive fiber 172 as an electrode wire 158—and a heating coil 110, which may be configured as a stretchable or non-stretchable conductive fiber. It should be appreciated, however, that the electrode of FIG. 14 may alternatively be configured as a temperature electrode.

The stretchable conductive fiber 172 of the ECG electrode 152 is woven into the mesh netting material 168 such that it extends laterally through the netting material 168 between a top surface 194 and a bottom surface 196 of the netting material 168 provided by the non-conductive stretchable fibers 170. Thus, the non-conductive stretchable fibers 170 insulate the stretchable conductive fiber 172 by spacing it apart from any adjacent conductive fibers or wires (not shown), such as the electrode wire of an adjacent electrode. The stretchable conductive fiber 172 extends through the netting material 168 from the sling cable (not shown) through the netting material 168 to the electrode contact 154. In the illustrated embodiment, the stretchable conductive fiber 172 extends up through the top surface 194 so that the stretchable conductive fiber 172 provides the contact surface 154 of the electrode 150. In some embodiments, he stretchable conductive fiber 172 may form a generally planar coiled shape, such as a spiraling disk, that can make contact with the neonate to acquire physiological signals or electrical potential. In some embodiments, however, at least one electrode may include an electrode contact plate or other member that is configured to contact the neonate and acquire physiological signals.

The heating coil 110, which may be configured as a stretchable or non-stretchable conductive wire, is woven into the netting material 168 such that the heating coil 110 is supported by a non-conductive stretchable fiber 170 below at a bottom surface 196 of the sling 160. At least one layer of non-conductive stretchable fiber 170 separates the heating coil 110 from the electrode wire 158, thereby electrically isolating the non-stretchable wire 174. Whereas the stretchable conductive wire 172 configured as an electrode wire 158 is tightly woven into the netting material 168, the heating coil may be loosely held so that it may shift relative to the surrounding stretchable fibers 170 of the netting material 168. This may be useful, for example, so that the netting material 168 can freely stretch and/or contract without bunching up or displacing heating coil 110 including a non-stretchable wire 174.

Although the embodiment of FIG. 14 depicts a sling 160 with conductive stretchable fibers 172 configured as electrode wires 158 and a heating coil 110 supported below the bottom surface 196, some embodiments may be differently configured. For example, a heating coil can be configured as a stretchable conductive fiber/wire that is woven into the netting material 168 between the top and bottom surfaces 194, 196 of the netting material 168. Additionally or alternatively, at least one of the electrode wires may be configured as non-stretchable conductive wire that is woven into the netting material 168 between the top and bottom surfaces 194, 196 of the netting material 168, and/or as a stretchable or non-stretchable conductive fiber/wire the is supported below bottom surface 196 of the netting material 168.

Further still, some embodiments of a sling may include at least one electrode wire or heating coil that is configured as a stretchable conductive fiber that takes the place of one of the laterally or longitudinally extending fibers 170 of the netting material 168. For example, referring to FIG. 14, at least one laterally extending non-conductive stretchable fiber 170 (e.g., fiber 170A) and/or at least one longitudinally extending stretchable fiber 170 (e.g., fiber 170B) may be replace with a stretchable conductive fiber that may be used as a heating coil or an electrode wire.

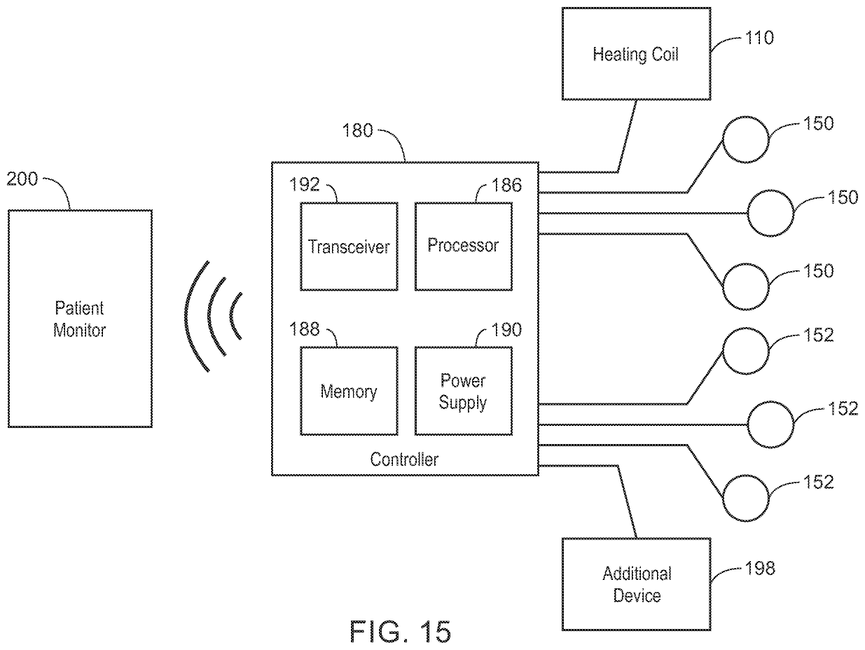

Referring to FIG. 15, a block diagram depicting the electronics of a neonatal sleep device 102 including a sling controller connected to a heating coil 110, temperature electrodes 150, and ECG electrodes is illustrated. It will be recognized that the sling controller 180 may include any of a variety of known controller circuits, integrated circuits, micro controllers, microprocessors, and associated circuity. The sling controller 180 may exemplarily include processor 186 with an integrated memory, a computer readable medium 188 for storing acquired signals and measurements, data, instructions and/or any other information, and a power supply 190 configured to power the sling controller 180, the heating coil(s)110, the drive electrode, and any other electrical components. The processor 186 is exemplarily configured to access software or firmware in the form of computer readable code stored on the computer readable medium 188 and/or in an integrated memory of the processor 186. The processor 186 may execute the computer readable code as an instruction set to carry out the functions as described herein, including the receipt of input, calculations, and outputs. The illustrated sling controller 180 additionally includes a transceiver 192 that is configured to wirelessly communicate information and/or instructions to an external patient monitor 200 or another computer system. Some embodiments, however, may include a sling controller 180 that communicates via a wired connection.

The sling controller 180 is connected to the heating coil 110, a plurality of temperature electrodes 150, and a plurality of ECG electrodes 152. The sling controller may additionally be configured to control an additional medical device 198. For example, the sleep device 102 may include a phototherapy device controllable by the sling controller 180. In such an embodiment, the phototherapy device may be secured to the frame 140 or positioned below or near the sling 160. Further still, some embodiments may include a phototherapy device that is integrated into the sling 160. For example, an electrotherapy device may include at least one light-carrying fiber that is woven into or supported by the netting material 168.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A bassinette for a neonatal care system comprising:
a frame structure including at least one vertical a support member extending downward from a top support member;
a sling suspended from the top support member and configured to support a neonate, the sling comprising a pressure-diffusing netting material extending from a top end to a bottom end between opposing lateral sides;
a heating coil configured to heat the neonate supported on the sling; and
a sling controller secured to the frame structure and configured to selectively control the heating coil to heat the neonate.

2. The bassinette of claim 1, wherein the heating coil extends in a serpentine pattern from a first end proximate the top end of the sling and a second end proximate the bottom end of the sling.

3. The bassinette of claim 1, wherein the heating coil is woven into the pressure-diffusing netting material of the sling.

4. The bassinette of claim 1, further comprising an array of temperature-sensing electrodes configured to acquire temperature measurements from a plurality of locations on the neonate; and
wherein the sling controller is configured to control the heating coil based on the acquired temperature measurements.

5. The bassinette of claim 1, wherein the heating coil is a first heating coil configured to heat a first portion of the sling, and wherein the bassinette includes at least one additional heating coil configured to heat a second portion of the sling.

6. The bassinette of claim 1, further comprising a cover configured to selectively enclose the neonate on the bassinette.

7. The bassinette of claim 6, wherein the cover is connected to the top support member and selectively retractable from a closed position in which the neonate is enclosed.

8. The bassinette of claim 1, further comprising a plurality of electrodes extending through the pressure-diffusing netting material of the sling and configured to acquire physiological signals from the neonate.

9. The bassinette of claim 8, wherein the sling is configured to communicate the acquired physiological signals to a patient monitor.

10. The bassinette of claim 8, wherein each electrode in the plurality of electrodes includes an electrode contact surface configured to make contact with the neonate and a stretchable wire linking the electrode contact surface to the sling controller.

11. The bassinette of claim 10, wherein the electrode contact surface of each electrode is provided by a portion of the stretchable wire configured to contact the neonate supported in the sling.

12. The bassinette of claim 8, wherein the heating coil is supported by the pressure-diffusing netting material below the plurality of electrodes such that the heating coil is spaced apart from the plurality of electrodes by non-conductive stretchable fibers of the pressure-diffusing netting material.

13. A sling configured to support a neonate in neonatal care system, the sling comprising:
a pressure-diffusing netting material extending from a top end to a bottom end between opposing lateral sides, wherein the top end, the bottom end, and the lateral sides are configured to be removably attached to a bassinette frame of the neonatal care system;
a plurality of stretchable conductive electrodes extending through the pressure-diffusing netting material and configured to acquire physiological signals from the neonate; and
wherein the plurality of stretchable conductive electrodes are configured to communicate the physiological signals from the plurality of stretchable conductive electrodes to a sling controller.

14. The sling of claim 13, wherein the plurality of stretchable conductive electrodes includes an array of temperature-sensing electrodes configured to acquire temperature measurements from a plurality of locations on the neonate.

15. The sling of claim 13, wherein the plurality of stretchable conductive electrodes includes an array of ECG electrodes configured to acquire electrocardiogram (ECG) signals from the neonate from a plurality of locations on a torso and head of the neonate.

16. The sling of claim 15, wherein the sling controller is configured to dynamically select at least one of the stretchable conductive electrodes to be used as a drive electrode to reduce signal noise in the acquired ECG signals.

17. The sling of claim 15, wherein the sling controller is configured to dynamically select at least two of the ECG electrodes in the array of ECG electrodes to acquire the ECG signals from the neonate.

18. The sling of claim 13, wherein the stretchable conductive electrodes are configured as stretchable conductive fibers that are woven into the pressure-diffusing netting material such that a plurality of stretchable non-conductive fibers of the pressure-diffusing netting material electrically isolate the stretchable conductive fibers of the stretchable conductive electrodes from each other.

19. The sling of claim 18, wherein a portion of at least one of the stretchable conductive fibers provides an electrode contact surface configured to make contact with the neonate supported in the sling.

20. The sling of claim 13, further comprising a heating coil woven into the pressure-diffusing netting material of the sling and configured to heat a neonate supported on the sling.

21. The sling of claim 20, wherein the plurality of stretchable conductive electrodes includes a stretchable electrode wire that is woven into the pressure-diffusing netting material and extends through the pressure-diffusing netting material; and
wherein the heating coil is supported by the pressure-diffusing netting material below the stretchable conductive electrodes such that the heating coil is spaced apart from the stretchable electrode wire by non-conductive stretchable fibers of the pressure-diffusing netting material.
